(19) United States
(12) Patent Application Publication
(10) Pub. No.: US 2010/0151554 A1
(43) Pub. Date: Jun. 17, 2010

(54) ENZYME COMPOSITION AND USE THEREFORE

(75) Inventors: Min-Cheng Wang, Taipei (TW); Raphael Chiou, Taipei (TW)

Correspondence Address: PAI PATENT & TRADEMARK LAW FIRM, 1001 FOURTH AVENUE, SUITE 3200, SEATTLE, WA, 98154, US

(73) Assignee: Ein Int'l Co. Ltd., Taipei (TW)

(21) Appl. No.: 12/636,734

(22) Filed: Dec. 12, 2009

(30) Foreign Application Priority Data
Dec. 15, 2008 (CN) 200810183778.5

(51) Int. Cl. C02F 3/34; C12N 9/02; C12N 9/10

(52) U.S. Cl. 435/189; 435/193; 435/262

(57) ABSTRACT

The present invention provides an enzyme composition and a method for treating coal using the enzyme composition. The enzyme composition includes at least one enzyme, coenzyme and ammonium acetate, wherein the enzyme can be laccase-isozyme, pyruvate dehydrogenase, dihydrolipoyl transacetylase, and dihydrolipoyl dehydrogenase, and the coenzyme can be CoA, CoA-SH, thiamine pyrophosphate, lipoic acid, flavin adenine dinucleotide, and nicotinamide adenine dinucleotide. In addition, the method of the present invention includes treating the coal with the enzyme composition for more than 72 hours.

### CROSS REFERENCE TO RELATED APPLICATIONS

This Application claims priority of China Patent Application No.200810183778.5, filed on Dec. 15, 2008 the entirety of which is incorporated by reference herein.

### BACKGROUND OF THE INVENTION

1. Field of the Invention

The present invention relates to a fermentation technology using an enzyme composition, and in particular relates to an enzyme composition for improving combustion efficiency of coals and decreasing the emission of NOₓ or SOₓ.

2. Description of the Related Art

Coal is a fossil fuel formed in ecosystems and composed primarily of carbon, hydrogen, oxygen with small quantities of nitrogen, sulfur or other elements. Sulfur is an important impurity in coals. Sulfur is usually changed to sulfur compounds after burning. In somes countries, for example, the United States, emission guidelines for sulfur compounds have already been established. Since it is expensive to remove pernicious elements, governments generally encourage using and producing low-sulfur-coals to decrease pollution.

According to the content of carbon, coal can be classificated into anthracite, semi-anthracite, bituminous coal, sub-bituminous coal, lignite, and peat.

In contrast with oil, coal is the largest source of fuel for the generation of world-wide electricity because coal is cheap. However, coal, in particular to peat, produces oxides of nigrogen and sulfur and smoke into the air to generate air pollution. These pollutents cause a coughing, asthma, and cancer in humans. Additionally, the oxides of nitrogen and sulfur can also cause acid rain.

In the beginning of the 20th century, Britain banned using raw coal. Currently, in order to protect the environment, factories using coal are either imposed a heavy air pollution tax or encouraged to close. Thus, a novel method and composition are needed to mitigate the previously mentioned problems.

### BRIEF SUMMARY OF THE INVENTION

The invention provides an enzyme composition, comprising at least one enzyme, at least one coenzyme, and ammonium acetate, wherein the enzyme comprises laccase-isozyme, pyruvate dehydrogenase, dihydrolipoyl transacetylase, or dihydrolipoyl dehydrogenase, and the coenzyme comprises CoA, CoA-SH, thiamine pyrophosphate, lipoic acid, flavin adenine dinucleotide, or nicotinamide adenine dinucleotide.

The invention provides a method for treating coal, comprising spraying the enzyme composition of the invention on the coals and treating the coals for more than 72 hours.

The invention also provides a method for improving the combustion efficiency of coals, comprising spraying the enzyme composition of the invention on the coals and treating the coals for more than 72 hours.

The invention further provides a method for decreasing the emission of oxides of nitrogen, sulfur, or carbon, comprising spraying the enzyme composition of the invention on the coals and treating the coals for more than 72 hours.

The invention further provides a method for decreasing hot corrosion, comprising spraying the enzyme composition of the invention on the coals and treating the coals for more than 72 hours.

The invention further provides a method for decreasing the production of coal cinders, comprising spraying the enzyme composition of the invention on the coals and treating the coals for more than 72 hours.

A detailed description is given in the following embodiments with reference to the accompanying drawings.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The present invention provides an enzyme composition. The enzyme composition comprises at least one enzyme, at least one coenzyme, and ammonium acetate, wherein the enzyme comprises laccase-isozyme, pyruvate dehydrogenase, dihydrolipoyl transacetylase, or dihydrolipoyl dehydrogenase, and the coenzyme comprises CoA, CoA-SH, thiamine pyrophosphate, lipoic acid, flavin adenine dinucleotide, or nicotinamide adenine dinucleotide.

In one embodiment, when a laccase-isozyme is selected as the enzyme, the coenzyme can be CoA-SH. In another embodiment, when the mixture of pyruvate dehydrogenase, dihydrolipoyl transacetylase, and dihydrolipoyl dehydrogenase is selected as the enzyme, the coenzyme can be CoA, thiamine pyrophosphate, lipoic acid, flavin adenine dinucleotide, and/or nicotinamide adenine dinucleotide.

The concentration of the enzyme of the present invention can be about ⅓ to 10/3×10⁻³ ppm.

The concentration of the ammonium acetate can be about 0.4 to 5 mg/L in the present invention.

The enzyme composition should be kept at or below 15° C., preferably, 4 to 10° C. However, it should be noted that, the enzyme composition cannot be kept below 0° C. to prevent inactivity of the enzyme composition.

The enzyme composition can be used to treat coals to improve the combustion efficiency of coals, decrease the emission of CO, NO, or SO, and suppress heat corrosion.

The “coals” of the invention refers to any coals. For example, the coals can be metallurgical coal or coking coal. The coals of the invention include, but are not limited to, anthracite, semi-anthracite, bituminous coal, sub-bituminous coal, lignite, or peat.

The coals can be a broken coals (<5 mm), or coal powders. The size of the coals is not limited, preferably, about 100 to 300 mesh. The efficiency of the enzyme composition can be improved depending upon decreasing the size of the coals.

The “oxides of sulfur or SOₓ” as used herein refers to a sulfur oxide (SO), sulfur dioxide (SO₂), and/or sulfur trioxide (SO₃).

The “oxides of nitrogen or NOₓ” as used herein refers to a nitrogen oxide (NO) and/or nitrogen dioxide (NO₂).

The “oxides of carbon or COₓ” as used herein refers to a carbon oxide (CO) and/or carbon dioxide (CO₂).

The present invention also provides a method for treating the coals. The method comprises spraying the enzyme composition of the invention on coals and then treating the coals for more than 72 hours. It should be noted that the coals are prevented from exposure to the sun when treated, and the treatment time exceeds 72 hours for complete interaction. The treatment temperature can be about 4° C. to 50° C., preferably, about 20° C. to 30° C.

In one embodiment, the coals are dried to decrease the relative humidity of the coals before treatment. The relative humidity can be controlled to be below 17% to improve the enzyme composition permeating into the coals.

The enzyme composition of the present invention can form an oxygen-rich film around the coals to decrease the ignition temperature and reduce the emission of COₓ (oxide of carbon) by more than 30%, preferably 30-40%, and promote complete combustion of the coals. In addition, the oxygen-rich film around the outside layers of coals can provide nascent oxygen to reduce the demand for fresh air resulting in the energy saving by more than 10%, preferably, 10 to 15%.

Further, the enzyme composition can reduce the emission of SOₓ and NOₓ. In one embodiment, the SOₓ can be reduced by more than 17%, preferably, 17%-50%. In another embodiment, the NOₓ can be reduced by more than 20%, more than 20-50%.

Moreover, the enzyme composition can split the coal molecules, cleave the organic long chain bond of the coals, and decompose the fibrous carbons, oil wax, gels, and impurities so that the coals can be completely burned. The combustion efficiency of the coals can be increased by more than 10%, preferably, about 10% to 15%. The amount of coal cinders can be decreased by more than 30%, preferably, about 30% to 50%. The enzyme composition can prevent the hot corrosion of equipment when burning

### EXAMPLE

### Example 1

### Preparation of the Enzyme Composition of the Invention

5 ml of 1 ppm laccase-isozyme and 50 g of ammonia acetate were added to 50 L of water and then completely mixed to obtain the enzyme composition of the invention. The enzyme composition was stored at 4° C. to 15° C. until use.

### Example 2

### Treatment of Coals Using the Enzyme Composition of the Invention

Coals were polished to become broken coals, wherein 80% of the broken coals could pass through a 200 mesh screen. The broken coals then were dried by heat to reduce the relative humidity to be below 17%.

The enzyme compositon produced from Example 1 was spraied on the dried coals, and the coals was mixed at 9, 12, and 17 o'clock, respectively. The coals was treated for 72 hours and prevented from direct exposure to the sun. After 72 hours of treatment, the coals were analysized. In the control group, the enzyme compositon was replaced with water.

### Example 3

### Combustion Test

The combustion test was accomplished by a SGS Group (Societe Generale De Surveillance). The results indicated that the enzyme composition of the invention could significantly reduce the emission of oxides of nitrogen and sulfur dioxide, wherein the oxides of nitrogen was decreased by 20%, and sulfur dioxide was decreased by 17% as shown in Table 1.

TABLE 1

| | Enzyme composition | Control group | Decrease |
|---|---|---|---|
| | (ppm) | (ppm) | rate (%) |
| Production of | 111 | 138 | 20 |
| oxides of nitrogen | | | |
| Production of | 7.8 | 9.4 | 17 |
| Sulfur dioxide | | | |

In addition, the combustion efficiency of coals and production of coal cinders were analysized. The results indicate that the enzyme composition could increase the combustion efficiency of coals by 14.0%, and decrease the production of coal cinders by 13.08%, as shown in Table 2.

TABLE 2

| | Enzyme composition | Control group | Decrease |
|---|---|---|---|
| | (ppm) | (ppm) | rate (%) |
| Production of coal | 20.07 | 23.09 | 13.08 |
| cinders | | | |

### Example 4

### Safety Test of the Enzyme Composition of the Invention

Rats were orally administrated laccase-isozyme of 1350 mg/kg body weight. After administration, the rats were active and did not appear to show signs of intoxication.

Cavies were orally administrated laccase-isozyme of 1352 mg/kg body weight per day for 28 days. After administration, the cavies were active and did not appear to show signs of intoxication.

A plate incorporation assay was used to test the safety of the laccase-isozyme. The laccase-isozyme did not cause any mutant of Salmonella and E. coli, after assay.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An enzyme composition, comprising at least one enzyme, at least one coenzyme, and ammonium acetate, wherein the enzyme comprises laccase-isozyme, pyruvate dehydrogenase, dihydrolipoyl transacetylase, or dihydrolipoyl dehydrogenase, and the coenzyme comprises CoA, CoA-SH, thiamine pyrophosphate, lipoic acid, flavin adenine dinucleotide, or nicotinamide adenine dinucleotide.

2. The enzyme composition as claimed in claim 1, wherein the coenzyme is CoA-SH when the enzyme is laccase-isozyme.

3. The enzyme composition as claimed in claim 1, wherein the coenzyme comprises CoA, thiamine pyrophosphate, lipoic acid, flavin adenine dinucleotide, and/or nicotinamide adenine dinucleotide when the enzyme is a mixture of pyruvate dehydrogenase, dihydrolipoyl transacetylase, and dihydrolipoyl dehydrogenase.

4. The enzyme composition as claimed in claim 1, wherein the enzyme has a concentration of about ⅓ to 10/3×10⁻³ ppm.

5. The enzyme composition as claimed in claim 1, wherein the ammonium acetate has a concentration of about 0.4 to 5 mg/L.

6. A method for treating coal, comprising spraying the enzyme composition as claimed in claim 1 on coals and then treating the coals for more than 72 hours.

7. The method as claimed in claim 6, further comprising mixing the coals after spraying the enzyme composition on the coals.

8. The method as claimed in claim 6, wherein the coals have a size of about 100 to 300 mesh.

9. The method as claimed in claim 6, wherein the coals are treated at a temperature below 50° C.

10. The method as claimed in claim 6, wherein the coals are prevented from direct exposure to the sun while treating.

11. The method as claimed in claim 6, wherein the coals comprise anthracite, semi-anthracite, bituminous coal, sub-bituminous coal, lignite, or peat.

12. The method as claimed in claim 6, wherein the coals comprise metallurgical coal or coking coal.

13. The method as claimed in claim 6, further comprising adding a glycerol or alcohol to the enzyme composition.

14. The method as claimed in claim 6, wherein the method improves the combustion efficiency of the coals.

15. The method as claimed in claim 6, wherein the method decreases the emission of SO, SO₂, and/or SO₃.

16. The method as claimed in claim 6, wherein the method decreases the emission of NO and/or NO₂.

17. The method as claimed in claim 6, wherein the method prevents the hot corrosion of equipment when burning.

18. The method as claimed in claim 6, wherein the method decreases the production of coal cinders.

19. A method for improving the combustion efficiency of coals, comprising spraying the enzyme composition as claimed in claim 1 on coals and treating the coals for more than 72 hours.

20. A method for decreasing the emission of oxides of nitrogen, sulfur, or carbon, comprising spraying the enzyme composition as claimed in claim 1 on coals and treating the coals for more than 72 hours.
